(19) United States
(12) Patent Application Publication
(10) Pub. No.: US 2016/0151202 A1
(43) Pub. Date: Jun. 2, 2016

(54) SYSTEM, METHOD AND ARRANGEMENTS FOR MODIFYING OPTICAL AND MECHANICAL PROPERTIES OF BIOLOGICAL TISSUES

(71) Applicant: THE GENERAL HOSPITAL CORPORATION, Boston, MA (US)

(72) Inventors: Giuliano Scarcelli, Cambridge, MA (US); Seok H. Yun, Cambridge, MA (US); Sheldon J.J. Kwok, Jamaica Plain, MA (US); Dieter Manstein, Coral Gables, FL (US)

(21) Appl. No.: 14/906,078

(22) PCT Filed: Jul. 21, 2014
(86) PCT No.: PCT/US14/47412

§ 371 Date: Jan. 19, 2016

Related U.S. Application Data
(60) Provisional application No. 61/856,479, filed on Jul. 19, 2013.

(51) Int. Cl. A61F 9/008

(52) U.S. Cl. CPC A61F 9/008; A61F 2009/00872

(57) ABSTRACT

System, method, arrangement and non-transitory computer-accessible can be provided for, e.g., effecting refractive changes of the cornea by spatially-selective two-photon crosslinking of collagen fibers. For example, it is possible to obtain at least one property of at least one portion of the eye using at least one first arrangement. Based on the at least one property, data indicating a plan of affecting the portion(s) of the eye can be generated. Further, it is possible to control at least one electromagnetic-radiation-providing second arrangement to execute the plan and irradiate the at least one portion based on the plan. The irradiation can be selectively controlled to be delivered to at least one selective depth within the portion(s).

### CROSS-REFERENCE TO RELATED APPLICATION(S)

The present application relates to U.S. Patent Application Ser. No. 61/856,479, filed Jul. 19, 2013, the entire disclosure of which is incorporated herein by reference.

### FIELD OF THE DISCLOSURE

Exemplary embodiments of the present disclosure relate to a modification of optical and mechanical properties of the tissues using nonlinear photochemical processes such as two-photon induced crosslinking, and more particularly to methods, systems, computer-accessible medium and arrangements that provide refractive changes of the cornea by spatially-selective two-photon crosslinking of collagen fibers. Further exemplary embodiments of the present disclosure relate to methods, arrangements and systems that can alter the structural, optical and mechanical properties of the corneal tissue using photochemical processes, such as, e.g., light-mediated crosslinking of collagen fibers.

### BACKGROUND INFORMATION

Collagen crosslinking is a procedure that involves a photosensitizing agent and light illumination that increases the strength of collagen fibers by inducing covalent crosslinks. Such procedure has become popular as a treatment for the cornea of patients affected by keratoconus or other ectatic disorders, i.e. conditions where the cornea is abnormally weak and therefore progressively thins and bulges.

Before 2003, therapeutic strategies for treating keratoconus included rigid gas-permeable contact lenses, thermal keratoplasty and intracorneal rings. However, all of these techniques aimed at managing the visual symptoms of keratoconus and were not able to arrest or even hinder the progression of keratoconus. In 2003, a clinical trial of Riboflavin/UVA cornea crosslinking (CXL) was reported for patients with moderate or advanced progressive keratoconus. (See, e.g., Ref 1).

CXL strengthens the cornea by inducing crosslinks among the collagen fibers of the corneal stroma. In all patients, the progression of keratoconus was arrested, and most patients experienced improved visual acuity. Subsequent studies confirmed these initial results and noted statistically significant improvements in long-term visual acuity. To date, CXL has been recognized as the only therapeutic approach that can arrest the progression of keratoconus.

The standard CXL protocol involves 1) epithelial debridement; 2) application of the photo sensitizer riboflavin (e.g., vitamin B2); and 3) irradiation with UVA light at 3 mW/cm² for 30 minutes. (See, e.g., Refs. 2 and 3). The mechanism of CXL is known to involve the production of singlet oxygen when riboflavin is bleached by UVA light. Singlet oxygen then catalyzes the formation of covalent cross-links between primarily histidine residues in collagen. No collagen cross-linking was observed when CXL treatment was performed in the presence of sodium azide, a singlet oxygen quencher. (See, e.g., Ref. 4). This can suggest that the production of singlet oxygen by riboflavin is a key step in collagen-cross-linking.

Recently, CXL has found other applications alone or in combination with other ocular procedures to alter the structural, mechanical and/or refractive properties of the cornea. The crosslinking procedure is therefore destined to become the standard of care for patients with corneal ectasia and is conceivable that its application will extend to other ocular tissues such as conjunctiva, sclera and possibly more broadly to other human tissues.

Despite the demonstrated usefulness of the standard “one-photon” collagen crosslinking of the cornea using UVA light and riboflavin (CXL) a few important drawbacks limit the widespread use and effectiveness of the procedure. For example, the significant thinning induced by the Riboflavin solution during the procedure and the fear of endothelial cell damage make standard CXL only applicable to thick corneas (greater than about 350 μm). In this respect, two-photon-induced processes in optical imaging, spectroscopy and microfabrication have advantages over their one-photon counterparts in part due to the spatial selectivity associated with two-photon processes. The invention makes use of two-photon or other nonlinear optical processes to selectively cross-link corneal collagen with 3-dimensional resolution. A two-photon cross-linking (2P-CXL) protocol allows treatment of thin corneas (less than about 330 μm) that cannot currently be treated, and can improve cell viability by using near-IR light and irradiating only non-cellular regions. In addition, selective collagen cross-linking can alter the refractive power of the cornea, for increasing visual acuity of the individual by correcting myopia or aberrations including high-order aberrations induced after refractive surgery or conventional corneal crosslinking. Since the two-photon cross-linking procedure can be readily customized, and furthermore is non-invasive and permanent, such an application could be a viable alternative to femtosecond laser ablation, or lamellar laser refractive surgery (LASIK).

It is believed that two-photon collagen cross-linking procedure (2P-CXL) of the cornea has not been reported or described. Traditional CXL of the cornea, using one-photon irradiation, is well established worldwide. 2P-CXL brings numerous advantages over the traditional CXL protocol in the treatment of keratoconus, including use of a less phototoxic near-IR laser, ability to selectively irradiate non-cellular regions only, and treatment of thin corneas that cannot be currently treated in the clinic.

A majority of the total refraction of the human eye is achieved by the cornea. (See, e.g., Ref 5). A number of refractive errors are due to abnormalities of the cornea, including astigmatism, hyperopia and myopia. In recent years, lamellar laser refractive surgery (LASIK) has emerged as an effective technique to modify the shape of the cornea and correct refractive errors associated with these disorders. In LASIK, a femtosecond laser is first used to etch a lamellar flap within the cornea stroma. The flap is then folded back, and an excimer laser is used to ablate and remodel the stroma. After ablation has been completed, the flap is repositioned to its original position, and left to heal naturally. Complications of LASIK include: lamellar keratitis induced by the femtosecond laser, dry eye due to severing of corneal nerves when the flap is created, displacement of the flap after surgery, and corneal ectasia, with similar clinical presentation of keratoconus.

U.S. Patent Publication No. 2010/0210996 describes a method for laser correction of refractive errors of an eye with a thin cornea by patterned crosslinking. Since this method relies on linear single-photon absorption of riboflavin, its spatial resolution, especially along the depth dimension, is inherently poor.

Another limitation of current CXL procedures is related to the requirement of de-epithelialization prior to the application of the drug. Human cornea has several layers, e.g., epithelium, Bowman's membrane, stroma, Descemet's membrane and endothelium. The stroma makes up the majority of corneal tissue and being rich in collagen fibers provides structural and mechanical strength of the cornea. CXL procedure aim to act on the corneal stroma and specifically on its collagen fibers.

The epithelial layer of the cornea, on the other hand, can be an extremely effective barrier against the diffusion of the photosensitizing agent into the corneal stroma. As CXL procedure's outcome is dependent on the effective delivery of the photosensitizing agent to the corneal stroma, by-passing the blocking function of the epithelium is truly a condicio sine qua non.

Standard protocols for collagen crosslinking address this issue by removing the epithelial layer prior to the application of the photosensitizer. For this reason, standard protocols can be generally referred to as “epi-off” CXL. However, removing the epithelium may have serious clinical drawbacks and side effects (see, e.g., Ref 21), e.g., (a) lengthening the post-operative recovery time, which may increase the pain involved with the procedure (that lasts about 5-7 days), (b) increasing the risk for infection, (c) losing corneal sensitivity for up to six months due to corneal nerve damage, (d) potential visual loss in the first days post-op, etc. Re-epithelialization takes a minimum of four days. It is typical that after CXL procedure antibiotics and steroids are prescribed for a week and patients need to be monitored during the first two weeks to assess corneal re-epitheliazation. (See, e.g., Ref. 21).

Thus, this is an important issue related to the CXL procedure, and an extremely active area of research. The clinical and research community has been exploring alternative procedures, usually referred to as “epi-on” procedures. The two major approaches currently investigated to achieve transepithelial delivery of the photosensitizer are, e.g. (a) Chemical loosening of the epithelial cell junctions (see, e.g., Ref. 22 and 23); and (b) Electrical driving of the photosensitizing solution into the cornea via iontophoresis (see, e.g., Ref 24). Both of these approaches have been shown to lack the necessary performance to replace the “epi-off” procedure; the evidence of their effectiveness is scarce and controversial, and therefore the clinical community has not adopted them. (See, e.g., Ref. 21). Although, as expected, the transepithelial “epi-on” delivery of photosensitizer reduces the side effects connected with epithelial debridment, the yield of delivery has been shown to be poor, which has produced decreased mechanical strengthening, and reduced clinical efficacy. (See, e.g., Refs. 21 and 25).

As a result, the growing field of collagen crosslinking has a clearly defined need to improve the delivery of the photosensitizer into the deeper layers of the cornea while not incurring in the clinical side-effects associated with the macroscopic removal of the epithelial layer. According to yet another exemplary embodiment of the present disclosure, the irradiation can be selectively controlled by the first arrangement(s) to provide a spatially-periodic pattern within the portion(s).

Accordingly, there may be a need to address at least some of the above-described deficiencies.

### SUMMARY OF EXEMPLARY EMBODIMENTS

Thus, to address at least such issues and/or deficiencies, exemplary embodiments of methods, systems, computer-accessible medium and arrangements that provide refractive changes of the cornea by spatially-selective two-photon crosslinking of collagen fibers can be provided. For example, using two-photon photochemical cross-linking to tune the refractive power of the cornea has not been reported. The exemplary embodiments of systems, methods and arrangement can provide a nonlinear corneal crosslinking provides a beneficial paradigm in the treatment of refractory disease without invasive ablation and creation of corneal flaps.

Thus, systems, methods, arrangements and non-transitory computer-accessible according to exemplary embodiments of the present disclosure can be provided for, e.g., affecting refractive changes of the cornea by spatially-selective two-photon crosslinking of collagen fibers. For example, it is possible to obtain at least one property of at least one portion of the eye using at least one first arrangement. Based on the at least one property, data indicating a plan of affecting the portion(s) of the eye can be generated. Further, it is possible to control at least one electromagnetic-radiation-providing second arrangement to execute the plan and irradiate the at least one portion based on the plan, affecting at least one property. The irradiation can be selectively controlled to be delivered to at least one selective depth within the portion(s).

The second arrangement(s) can include a laser source configured to excite multi-photon transitions. The laser source can be a pulsed femto-second laser source which can be configured to deliver near an infra-red light radiation. The propert(ies) can include (i) refractive index, (ii) elastic or visco-elastic property, (iii) microstructure, (iv) radius of curvature, (v) collagen content and organization, and/or (vi) scattering effect of the at least one portion. The propert(ies) can be obtained by (i) OCT, (ii) Brillouin imaging modality (iii) Raman, (iv) laser speckle, (v) multi-photon imaging modality, (vi) photo-acoustic modality, (vii) confocal microscopy modality, (viii) florescence modality, and/or (ix) pentacam. For example, the change(s) can effect at least one optical property of the eye. The optical propert(ies) can include (i) a refractive property, (ii) a transmission property, (iii) a polarization filter, (iv) a reflection property, (v) a color filter, and/or (vi) a refractive error within the eye. The refractive error can include a myopia, a hyperopia, an astigmatism and/or a high-order aberration. The high-order aberration can include a spherical aberration and/or a coma aberration.

According to an exemplary embodiment of the present disclosure, the irradiation can be delivered to a specifically controlled volume within the portion(s), e.g., without effecting further sections of the portion(s) through which the irradiation is provided. The specifically controlled volume can be as small as a diffraction-limited spot delivered by the second arrangement up to less than the volume of the portion(s). For example, the specifically controlled volume can be approximately 1 μm³.

According to another exemplary embodiment of the present disclosure, at least one third arrangement can be provided which is configured to affect a further property of the eye (i) prior to and/or (ii) during the delivery of the irradiation to the portion(s). For example, the arrangement(s) can be configured to applanate the cornea or counteract the intrinsic refractive power of the cornea to facilitate cross-linking, and includes at least one of (i) a contact lens (ii) a concave lens, (iii) a convex lens, (iv) an applanating transparent window or (v) a prism. The portion(s) can contain a photo-activatable agent. The first arrangement(s) can activate the photo-activatable agent so as to cause a selective cross-linking. The first arrangement(s) can utilize the selective cross-linking to treat keratoconus in the portion(s). Alternatively or in addition, the first arrangement can obtain information regarding keratoconus in the portion(s), and can be used to change a refractive property of the portion(s) based the information using the selective cross-linking. The refractive property can include a high-order aberration, and the high-order aberration can include a spherical aberration and/or a coma aberration.

Further, or in addition, upon the execution of the plan and the delivery of the irradiation to the at least one portion based on the plan, (i) a refractive error and/or (ii) an imperfection of the eye can be improved, and/or at least one separation within the eye can be reconnected.

Turning to another exemplary embodiment of the present disclosure, a first step of the above-described CXL procedure, i.e. the de-epithelialization of the cornea prior to the procedure, can be modified. Thus, such exemplary embodiments can be broadly applicable to different photosensitizers and illumination strategies for CXL.

Thus, system, method and arrangement according to such exemplary embodiment of the present disclosure can address the issue of improving a delivery of the photosensitizer into the deeper layers of the cornea while not incurring in the clinical side-effects associated with the macroscopic removal of the epithelial layer. Thus can be done, e.g., by introducing microscopic spatially patterned debridement of the epithelium. Removing and/or ablating small localized zones of the epithelial layer can leave intact enough surrounding normal tissue which facilitates very quick re-epitelialization. Further, the localized “micro-holes” created in the epithelium can enhance a diffusion of the photosensitizing agent through the epithelium into the stroma.

Thus, according to certain exemplary embodiments of the present disclosure, an inscribing arrangement can be provided to produce microscopic injury with a pattern on the epithelium of a cornea. Such exemplary arrangement can include, e.g., a micro-needle array, an optical arrangement to generate at least one pattern, energy source (e.g., a pulsed laser, such as, e.g., femtosecond laser), mask/scanner, lens, etc. Various patterns can be achieved with such exemplary configuration (e.g., array pattern, patterned area, spacing, diameter, shape, depth, etc.).

According to yet another exemplary embodiment of the present disclosure, a drug delivery system can be provided which can include the above described arrangement, and utilize certain exemplary and chemical agents. Such exemplary agents can include certain chemical agents delivered/diffused through the injury produced by the tool, Riboflavin, Photosensitizer and/or eye drugs.

In still a further exemplary embodiment of the present disclosure, an apparatus for corneal treatment by microscopic epithelium debridement can be provided. Such exemplary apparatus can utilize the following, e.g., CXL, including CXL light, refractive correction, CXL light, and/or PDT, excitation light. A chemical agent can be used with chemical structure optimized for an efficient transport through microscopic debridement. The exemplary properties of the chemical agent can be further optimized to work in combination with other strategies, such as iontophoresis, for more rapid and effective diffusion.

These and other objects, features and advantages of the present disclosure will become apparent upon reading the following detailed description of exemplary embodiments of the present disclosure, when taken in conjunction with the appended drawings and claims.

### BRIEF DESCRIPTION OF THE DRAWINGS

Further objects, features and advantages of the present disclosure will become apparent from the following detailed description taken in conjunction with the accompanying drawings showing illustrative embodiments of the present invention, in which:

FIG. 1 is a schematic diagram of an exemplary embodiment of the apparatus to perform nonlinear crosslinking of the cornea;

FIG. 2A is a cross-sectional view of an exemplary cross-linked pattern generated by the exemplary progression according to an exemplary embodiment of the present disclosure;

FIG. 2B is a top view of the exemplary cross-linked pattern of FIG. 2A;

FIG. 2C is an exemplary refractive index profile of a substantially periodic and/or a chirped grating with an enhanced reflection of an ultraviolet (UV) light;

FIG. 3A is an illustration of the eye with exemplary aberrations;

FIG. 3B is an illustration of the treated eye with corrected aberrations by the exemplary system, method and/or arrangement (e.g., 2P-CXL) according to an exemplary embodiment of the present disclosure;

FIG. 3C is an illustration of the eye (e.g., relaxed state) with myopia;

FIG. 3D is an illustration of the treated eye with corrected aberrations by the exemplary system, method and/or arrangement (e.g., 2P-CXL) according to an exemplary embodiment of the present disclosure;

FIG. 4 is an illustration of an exemplary second-harmonic generation micrograph of the corneal tissue after the exemplary 2P-CXL, showing collagen fibers;

FIG. 5 is a set of illustrations that illustrate an exemplary measurement configuration to detect the change in refractive focus of cornea as performed by the system, method and arrangement according to the exemplary embodiment of the present disclosure

FIGS. 6A and 6B are exemplary phase contrast and fluorescent micrographs, respectively, of a 2P-CXL cross-linked volume in the cornea obtained with the exemplary system, method and/or arrangement (e.g., 2P-CXL);

FIG. 7 is a graph depicting changes in riboflavin fluorescence, collagen second harmonic generation and tissue autofluoresence during the exemplary procedure performed by the exemplary system, method and/or arrangement (e.g., 2P-CXL)

FIG. 8 is a set of cross-sectional views of a cornea undergoing exemplary procedures executed by the system, method and apparatus according to another exemplary embodiment of the present disclosure;

FIG. 9 is a set of illustrations of various exemplary configurations of an application of electro-magnetic radiation with at least some portions of the exemplary apparatus for producing patterned epithelium debridement according to the exemplary embodiments of the present disclosure; and

FIG. 10 is sets of exemplary patterns used with the exemplary embodiments of the present disclosure for impacting the cornea, and the exemplary results of such applications in accordance with the exemplary embodiments of the present disclosure.

Throughout the drawings, the same reference numerals and characters, if any and unless otherwise stated, are used to denote like features, elements, components, or portions of the illustrated embodiments. Moreover, while the subject disclosure will now be described in detail with reference to the drawings, it is done so in connection with the illustrative embodiments. It is intended that changes and modifications can be made to the described exemplary embodiments without departing from the true scope and spirit of the subject disclosure and the appended claims.

### DETAILED DESCRIPTION OF EXEMPLARY EMBODIMENTS

An exemplary production of singlet oxygen by riboflavin occurs as follows. Here, we describe riboflavin as the photo-initiator, but there are several photo-initiator dyes known in the fields, including riboflavin derivatives and Rose Bengal, which has single-photon absorption in the green wavelength. Excitation of riboflavin can be accomplished with one-photon absorption, with use of UVA light (315-400 nm), or two-photon absorption (2PA) with use of a femtosecond laser that delivers near-IR light (800 nm). Although 2PA can follow different selection rules, two-photon photosensitized production of singlet oxygen has been demonstrated. (See, e.g., Refs. 7 and 8). 2PA generally relies on the simultaneous absorption of two photons, each with half the energy that is used in the one-photon process. A spatial selectivity of 2PA can arise because the probability of absorption is dependent on the square of the incident laser power. In addition, 2PA requires extraordinarily high peak laser intensities. As a result, two-photon absorption is confined to the laser focus (Δx<1 μm), which can minimize an excitation of molecules in the out-of-focus regions. This phenomenon contrasts with one-photon absorption, which can be linearly dependent on incident laser intensity, and can occur throughout the incident light cone.

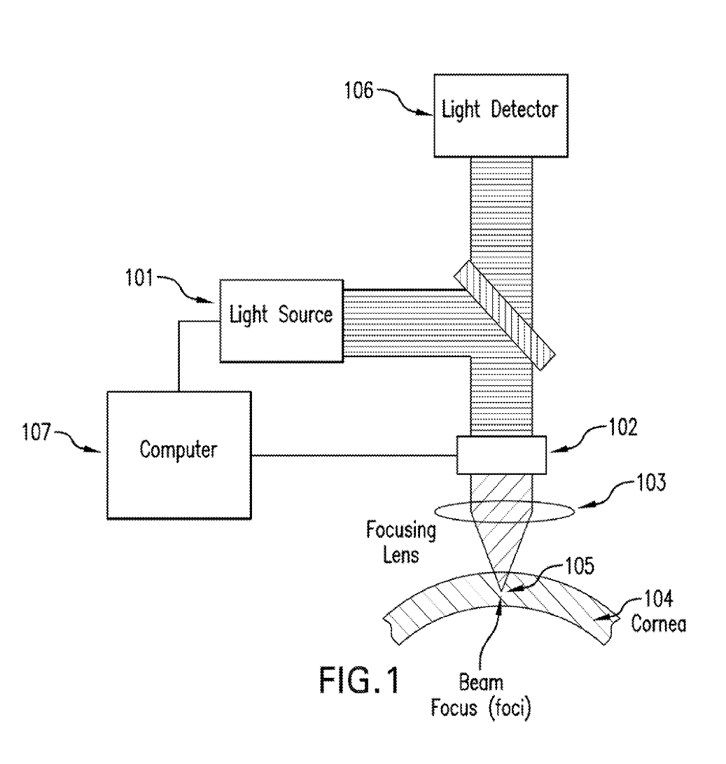

FIG. 1 shows a schematic diagram of a procedure for nonlinear crosslinking of the cornea according to an exemplary embodiment of the present disclosure. As shown in FIG. 1, excitation light can be provided by a light source (101). The light source can be, but not limited to, a mode-locked femtosecond laser capable of providing, e.g., 100-300 fs duration in the red or near infrared wavelengths. Indeed, there is a wide range of light sources suitable for 2P-CXL depending on specific requirements. 2P-CXL utilizes a spatial control of the optical intensity, which can be achieved by employing, e.g., a 2 to 3-axis beam scanner (102) and focusing lens (103), and/or a combination of spatial light modulator or deformable mirrors. Specific crosslinking patterns can be written or generated within the cornea (104) by controlling the duration and/or intensity of the focused writing beam at each spatial location (105). To evaluate the specific crosslinking pattern needed for a specific application, the same light source (101) or a different source of radiation can be used to measure the properties of the cornea with a light detector (106) or another detector that is configured to detect electro-magnetic radiation. Several known optical or non-optical modalities can be used for this purpose, such as imaging modalities including, but not limited to, e.g., confocal microscopy, phase-contrast imaging, adaptive optics imaging, optical coherence tomography, photoacoustic imaging or ultrasound; or spectroscopy modalities such as Brillouin, Rayleigh or Raman spectroscopy. The information regarding the cornea is analyzed by a computer (107) which can be specifically programmed determines the spatial pattern of the crosslinking procedure. The same or additional light source(s), and the same or additional light detector(s) can be also used to monitor the outcome of the CXL procedure, for example with linear or non-linear microscopy.

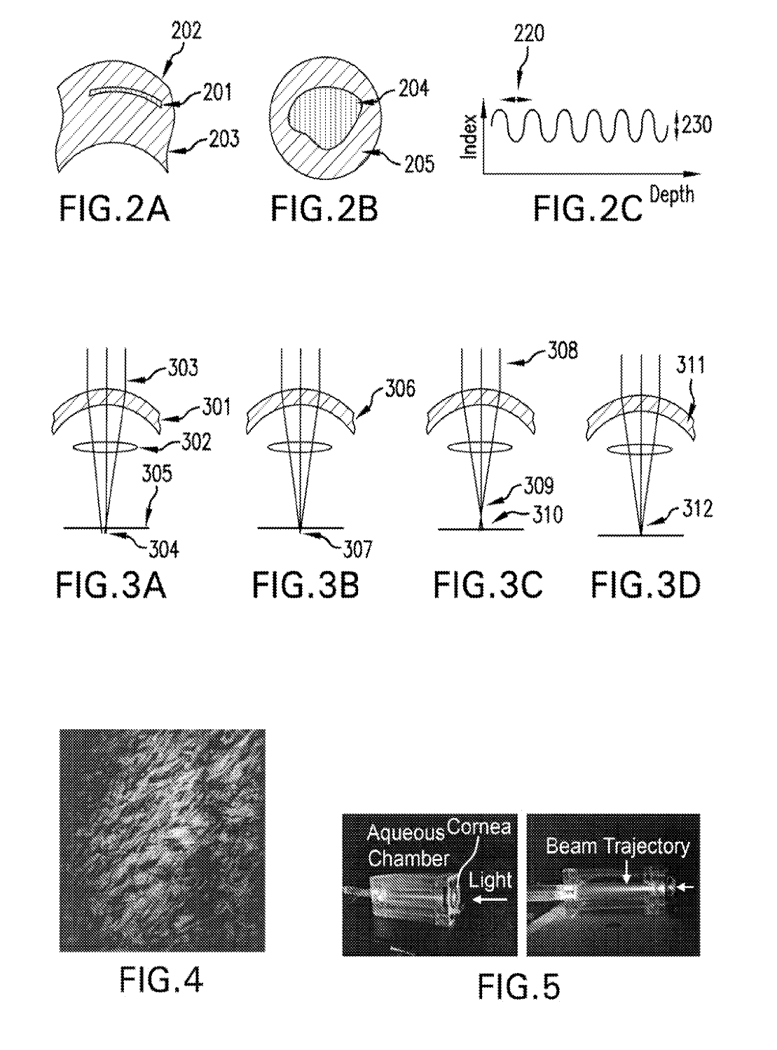

FIGS. 2A-2C shows exemplary crosslinking patterns. For example, in FIG. 2A, an exemplary crosslinking pattern (201) can be produced at a specific depth beyond the epithelium of the cornea (201) within the stroma (203). In the top view of FIG. 2B, a crosslinked pattern (204) also show lateral selectivity, leaving a portion of the cornea untreated (205). The crosslinking patterns can depend on a particular or specific objective of the procedure. For example, while normal corneas present a largely uniform elastic modulus in exemplary lateral directions; in keratoconus patients, we have measured the lateral spatial distribution of the corneal modulus to be very asymmetric with the region around the cone abnormally weakened and the regions outside of the cone close to normal. In an exemplary axial direction, the rigidity of the normal stroma can decrease through the depth with a light slope in the anterior stroma and steeper slope in the posterior stroma; on the other hand, keratoconus corneas show a very rapid decrease of rigidity through depth. Given this scenario, the 3D spatial selectivity enabled by 2P-CXL can, e.g., be used to target the anterior or posterior regions for maximal effectiveness, and/or increase the modulus only in abnormally weakened regions. The measurement of the local mechanical properties is one example of the potential corneal properties that can be used as feedback mechanism to calculate a specific crosslinking pattern. In general, the exemplary pattern can be determined to maximize the visual acuity and adequate corneal stiffness. Other exemplary factors, such as an exemplary minimal procedure time, can be considered and/or utilized.

In general, crosslinking can result in a local change of the refractive index. This can be used to produce a spatial refractive grating in the cornea. An exemplary pattern is shown in FIG. 2C, where the refractive index is modulated periodically as a function of depth. The periodicity 220 can be substantially uniform in space, or strategically made aperiodic or chirped. The modulation depth 230 can also be substantially uniform in space or has a gradient. Such exemplary pattern can be achieved, for example, by scanning the focus of the writing beam.

2P-CXL can provide certain important benefits over standard CXL treatment. First, a prolonged illumination of corneas with UVA light in CXL can cause cell death and tissue toxicity. 2P-CXL can use a near-IR laser at about 810 nm, which is less phototoxic. (See, e.g., Ref 9). Second, the 2P-CXL procedure can be tailored to avoid keratocytes in the cornea, which are known to disappear from the anterior stroma soon after CXL treatment with UVA irradiation. (See, e.g., Ref 2). While the keratocytes can eventually repopulate the anterior stroma, the effects of their initial insult on cornea morphology may not be well understood. CXL treatment can also stimulate an inflammatory cell activation in the corneal stroma. At present, the long-term effects of CXL treatment on the cornea may not be known, due to a lack of long-term follow-up studies. By minimizing or reducing cell damage with 2P-CXL, long-term side-effects can be mitigated. Third, the 2P-CXL procedure would allow treatment of thin corneas (<330 μm) that cannot be treated with current CXL procedures. Typically, CXL treatment requires a minimal corneal thickness of 400 μm to prevent damage to the underlying endothelium. An exemplary protocol, which can use a hypo-osmolar riboflavin solution, can induce swelling of the cornea thereby extending this limit, while the protocol still uses a thickness of 330 μm. Since 2PA generally occurs at the laser focus, which is typically less than about 1×1×4 μm³, 2P-CXL can treat thin corneas without damaging the endothelium beneath the stroma. Fourth, 2P-CXL can be used in conjunction with Brillouin microscopy (see, e.g., Ref 11 and 12), or other imaging modalities that can provide spatially-resolved information regarding the local properties, optical and/or mechanical, of the cornea to select and cross-link corneal areas most distorted by keratoconus, thereby minimizing the overall irradiation.

An exemplary ability to selectively cross-link the cornea can be used for certain applications, e.g., in modifying the refractive power of the cornea. The cornea can be modeled as a simple two-surface optical system and/or a positive meniscus lens. The refractive power can be calculated and/or determined with the refractive indices of the multiple regions (e.g., air, cornea, aqueous), and the radii of curvature of the anterior corneal surface and the posterior corneal surface 13. By altering the radius of curvature of the cornea globally or locally, e.g., on the anterior surface, with 2P-CXL, the refractive power of the cornea can be altered. The refractive index and thickness of cornea (stroma) can also be altered by 2P-CXL procedure. These and other exemplary physical and chemical effects can be considered when determining the exemplary crosslinking pattern. The incident laser power, cross-linking duration, photosensitizer concentration can also be optimized to achieve the desired effect of 2P-CXL.

Abnormal shape of the cornea can result in refractive errors such as low-order aberrations including myopia, hyperopia and astigmatism, and high order-aberrations such as spherical aberrations and coma. FIG. 3A shows an example of an eye whose cornea (301) and lens (302) do not yield perfect vision and present certain aberrations so that incoming collimated rays of light (303) are not focused to a tight point (304) onto the retina (305). In this exemplary case, after the 2P-CXL procedure, the treated cornea (306) can restore perfect vision (or at least near to that), and focus to a tight spot (307). In another example, shown in FIG. 3C, in the case of myopia, an incoming collimated light (308) or other electromagnetic radiation can be focused before the plane of the retina (309), so that the retina sees a blurred spot (310). Spatially patterned crosslinking can facilitate the correction of this focusing error by, e.g., modifying the local and global mechanical and refractive properties of the cornea, as shown in FIG. 3D, where the treated cornea (311) has perfect focus onto the plane of the retina (312). For different refractive errors, exemplary spatial patterns can be provided which can include, e.g., a ring or a circle at a specific depth for focal length errors, and asymmetric patterns for astigmatisms.

The exemplary system, method and/or arrangement (e.g., 2P-CXL) according to various exemplary embodiments of the present disclosure can also be performed at a low enough power such that the major effects of the procedure are alterations in the cornea's refractive index rather than changes in curvature. Spatially patterned crosslinking at low power can also be used for higher order aberrations, which is a common side-effect of LASIK.

In addition, 2P-CXL can facilitate a modulation of the refractive index within the cornea in a periodical, quasi-periodical, or a-periodical manner in 3D. This can empower the eye with color filtering, polarization filtering (e.g., by modulating birefringence) and visual acuity previously not possessed by the eye. For example, the 2P-CXL procedure can induce a local increase in refractive index from the natural stroma value of 1.37 to up to 1.5. In this case, the periodic modulation of refractive index within parallel thin layers (e.g. N>10) of the corneas (see, e.g., FIG. 2C) can produce selective reflection of a wavelength of light or a bandwidth of wavelengths. The optimal periodicity (220) can be equal to the half of the central wavelength of the reflection band. For example, an index grating with a periodicity of 250 μm can reflect a UV band centered at about 250 μm. If it is difficult to achieve the periodicity due to the limited optical resolution of the writing beam, it is possible to provide and/or facilitate the periodicity to be an integer multiple of the wavelength so that one of the spatial harmonic frequencies satisfies the Bragg reflection condition. This exemplary arrangement can be beneficial, for example, to avoid harmful UV radiation to reach the crystalline lens and/or the retina, thus likely reducing the risk to develop cataracts and retinal photo-chemical damage.

An exemplary ability to alter with spatial selectivity the radius of curvature and refractive index of the cornea, allows the exemplary 2P-CXL technique to take advantage of diagnostic and structural information provided by existing measurements and/or modalities of the cornea. These can include, but not limited to, optical coherence tomography (OCT), pentacam and numerous imaging techniques such as laser speckle, Raman, photo-acoustic, multi-photon, photo-acoustic and fluorescence. Information provided by these measurements before the exemplary techniques of 2P-CXL to be performed can be used to generate an optimized three-dimensional plan for cross-linking the patient's eye to correct the patient's vision. For example, an exemplary 2P-CXL procedure can include: a) an exemplary analysis of three-dimensional OCT image and/or a Pentacam of the patient's cornea demonstrating myopia caused by excessive curvature in the anterior portion of the cornea, b) an exemplary computation/determination of an exemplary pattern used and/or required to correct patient's vision (e.g. a ring around the anterior portion of the cornea) and of the operational parameters which can be used to achieve the desired changes; c) an exemplary application of riboflavin to the cornea, and/or d) an exemplary two-photon cross-linking of a ring around the anterior portion of the cornea, at an optimized depth within the anterior portion of the cornea, e.g., to flatten or reverse the excessive curvature and restore optimal vision.

The exemplary embodiment of the system, method and arrangement according to the present disclosure can further utilize and/or include monitoring device(s). For example, photobleaching of riboflavin two-photon fluorescence during the exemplary 2P-CXL procedure, as shown in FIG. 7, can be a useful real-time indicator of the exemplary 2P-CXL efficacy. For more direct measurements, for example, biomechanical rigidity, measured by the elastic modulus, can accurately report CXL treatment effectiveness on the cornea. (See, e.g., Ref 2). The Young's modulus of porcine and human corneas can be increased by a factor of, e.g., about 1.8 and 4.5 respectively with CXL treatment. (See, e.g., Ref 14). Shear modulus can be measured by shear-strain measurements using, e.g., a stress-controlled rheometer. Confocal Brillouin microscopy, an alternative method of measuring biomechanical rigidity, is now being used routinely in our laboratory. (See, e.g., Refs. 11 and 12). This exemplary technique can rely on the scattering of incident photons with propagating thermodynamic fluctuations known as acoustic phonons. The resulting exemplary frequency shift, known as the Brillouin shift, can be related to the longitudinal elastic modulus of the sample. Brillouin microscopy has been used for non-invasive three-dimensional imaging of cornea rigidity and to evaluate changes in rigidity with CXL treatment. (See, e.g., Refs. 12 and 15). These exemplary tools and/or techniques can be used with or integrated into the exemplary 2P-CXL procedure, system and arrangement according to an exemplary embodiment of the present disclosure to assess the efficacy or monitor the progression of crosslinking during procedure. Additionally, optical characterization, such as topography or optical coherence tomography, can also be used to assess and monitor the refractive changes and visual acuity.

To demonstrate the feasibility of the exemplary 2P-CXL procedure, system and arrangement according to an exemplary embodiment of the present disclosure, two-photon photobleaching experiments have been performed on riboflavin. For example, a Ti:Sapphire femtosecond laser delivering 810 nm, 150-fs, 80 MHz pulses was used to measure the reduction of riboflavin fluorescence over time. It was confirmed that riboflavin could be bleached using two-photon photoactivation. The photobleaching rate can be expected to depend quadratically on the laser power, much like two-photon absorption (I˜powerⁿ, where n=2). It was also determined that the photobleaching rate were dependent on even higher order photon interactions (n>2). Similar results with other fluorophores have been reported, although the mechanism of these higher order interactions is not well understood. (See, e.g., Ref 16). Nevertheless, the added non-linearity of two-photon induced riboflavin photobleaching provides superior spatial selectivity for the purposes of 2P-CXL.

Furthermore, a two-photon crosslinking procedure of porcine corneas has been performed in accordance with the exemplary embodiments of the present disclosure using an exemplary beam-scanning illumination system according to an exemplary embodiment of the present disclosure. FIG. 4 shows an illustration of an exemplary second-harmonic generation micrograph of the corneal tissue after the exemplary 2P-CXL, showing collagen fibers. For example, a laser beam was raster scanned over the field of view.

To determine the focal length of the cornea, according to one experiment, an excised ex vivo bovine cornea was mounted on a custom-made transparent aqueous chamber (see FIG. 5). Due to this chamber, it is possible to measure the refractive focus of cornea before and after a certain exemplary procedure. This facilitates a quantification of the overall optical effects on the cornea as performed by the system, method and arrangement according to the exemplary embodiment of the present disclosure. For example, water pressure was applied to prop the cornea up and maintain its shape. A laser incident on a fluorescent solution was translated up and down to trace rays visualizing the focus of the cornea.

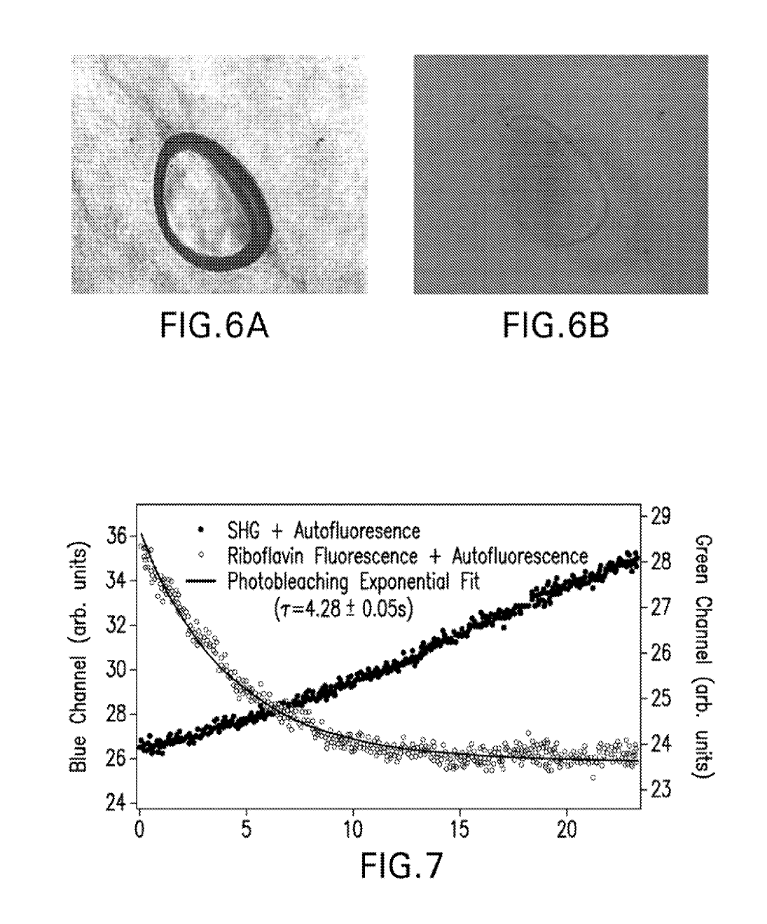

FIGS. 6A and 6B shows exemplary phase contrast and fluorescent micrographs of the exemplary 2P-CXL treated corneas taken by a conventional inverted microscope. The magnification of both images are, e.g., 2 by 1.5 mm. In this experiment, an elliptical region of interest was cross-linked throughout the depth of the cornea. The black halo in FIG. 6A is likely due to a change in refractive index at the interface of non-crosslinked and crosslinked tissue. FIG. 6B shows photobleaching of riboflavin in the region that was crosslinked by 2P-CXL.

FIG. 7 shows s graph of exemplary time dynamics of collagen second harmonic generation (darker shade channel) and riboflavin two-photon fluorescence (lighter shade channel) as a function of time during the exemplary two-photon procedure. For example, in both channels, a tissue autofluoresence background is observed. The riboflavin fluorescence bleaches over time, as expected, and is a good fit to an exponential function. The exemplary laser power can be adjusted to increase or decrease the duration of the exemplary 2P-CXL irradiation.

2P-CXL remodeling of the cornea according to an exemplary embodiment of the present disclosure can be a suitable alternative to (or used in addition to) LASIK, since the exemplary procedure(s) which can be implemented by such exemplary embodiments is non-invasive, permanent and customizable for the patient's needs. The exemplary systems, methods and arrangements according to the present disclosure which utilize 2P-CXL do not require ablation of tissue or creation of a corneal flap. In comparison to LASIK, 2P-CXL delivers much lower peak laser intensities are delivered to the tissue. For example, in femtosecond laser cutting of corneal flaps, 2-3 μJ can be delivered per laser pulse (see, e.g., Ref. 17), while only 0.75 nJ can be delivered per laser pulse with use of our 2P-CXL procedure, assuming an average laser power of about 60 mW and repetition rate of about 80 MHz. In cases where LASIK may be preferable, 2P-CXL can also be used in conjunction with LASIK for the purposes of selective corneal flap bonding since the flap can be displaced after LASIK surgery.

CXL treatment with riboflavin is used clinically. The 2P-CXL protocol is relatively simple, and does not require expensive equipment other than a femtosecond laser, which is often available for clinical use. For these reasons, the exemplary systems, methods and arrangement of the present disclosure which implement 2P-CXL can be effective, and used not only for the treatment of keratoconus, but also as a viable alternative to LASIK for the treatment of various refractive disorders such as astigmatism, hyperopia and myopia.

In addition to collagen crosslinking, the exemplary systems, methods and arrangement of the present disclosure may utilize different nonlinear processes, such as, e.g., two-photon induced local release of chemicals. For example, molecules can be encapsulated by nano carriers, such as hollow gold nano-cubes coated with thermally sensitive polymers (see, e.g., Ref. 18), and illumination of femtosecond pulses release the molecules, such as collagenase, to induce physical and chemical changes of the cornea.

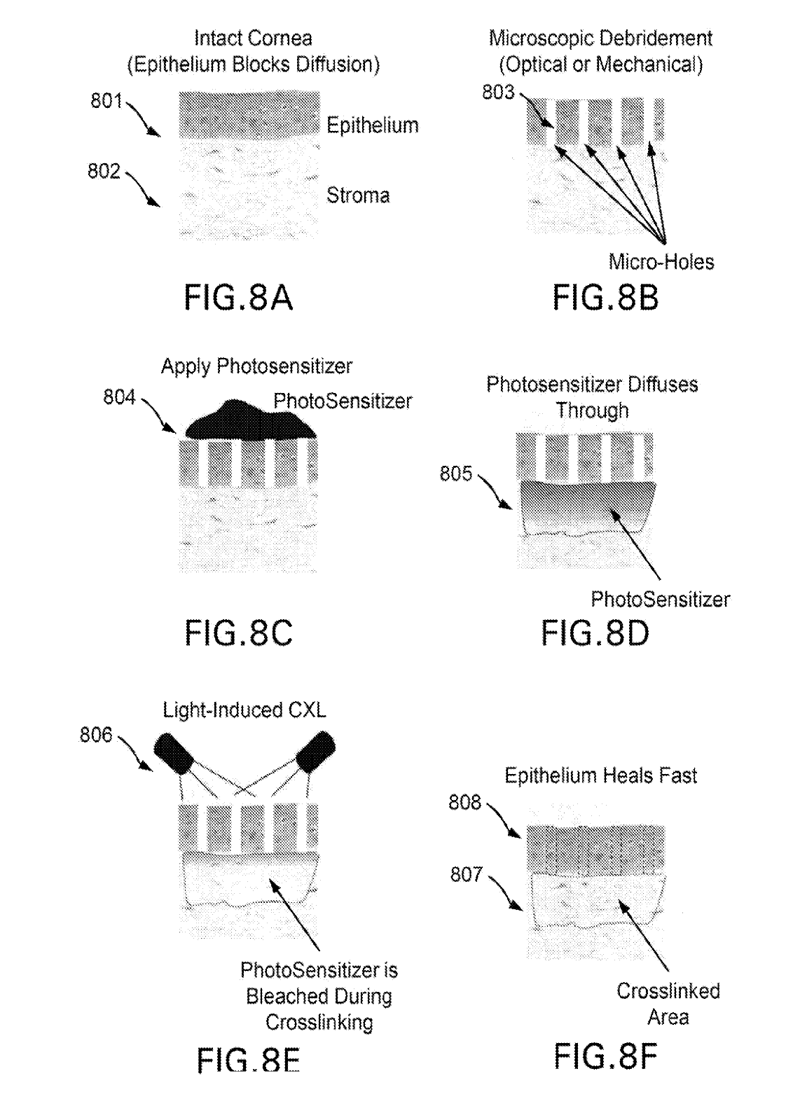

FIG. 8 shows a set of cross-sectional views of a cornea undergoing exemplary procedures executed by the system, method and apparatus according to another exemplary embodiment of the present disclosure. In this exemplary embodiment, the epithelium (801) of the cornea (see, e.g., FIG. 8A), a layer that is known to clock the diffusion of photo-activatable agents through the stroma (802), is not totally removed as in standard CXL procedure, and is partially debrided by creating microholes (803) with mechanical, or optical methods (see, e.g., FIG. 8B). After the exemplary debridement procedure, a photosensitizer (804) can be applied which can easily diffuse through the stromal tissue (805), as shown in FIGS. 8C and 8D. At this stage, light (806) or other electromagnetic radiation can be applied to the corneal tissue to induce photochemical crosslinking (see, e.g., FIG. 8E). After the exemplary procedure, it is likely that the stroma has been fully crosslinked (807) and the microscopic debridement facilitates a faster corneal healing (808) than a traditional CXL procedure (see, e.g., FIG. 8F). Faster healing and absence of scars involved with micro-injuries, micro-removals and/or micro-ablation is described in the literature in skin, where much deeper removal or injuries have been tested. (See, e.g., Ref. 26). On the other hand, the yield of delivery of the photosensitizing agent through the “micro-holes” can be expected to depend on the fraction of the surface area that can be ablated/removed while still maintaining enough untreated tissue to enable fast re-epithelialization; in the publications regarding skin, it has been shown that up to 20-50% of surface area can be removed while still maintaining fast re-epithelialization and avoiding scar formation. (See, e.g., Ref 27). Microscopic debridement of the epithelium can be obtained with microscopic needles, or robotically automated/multiplexed microbiopsy punches. Alternatively, epithelium debridement can be achieved optically through laser ablation.

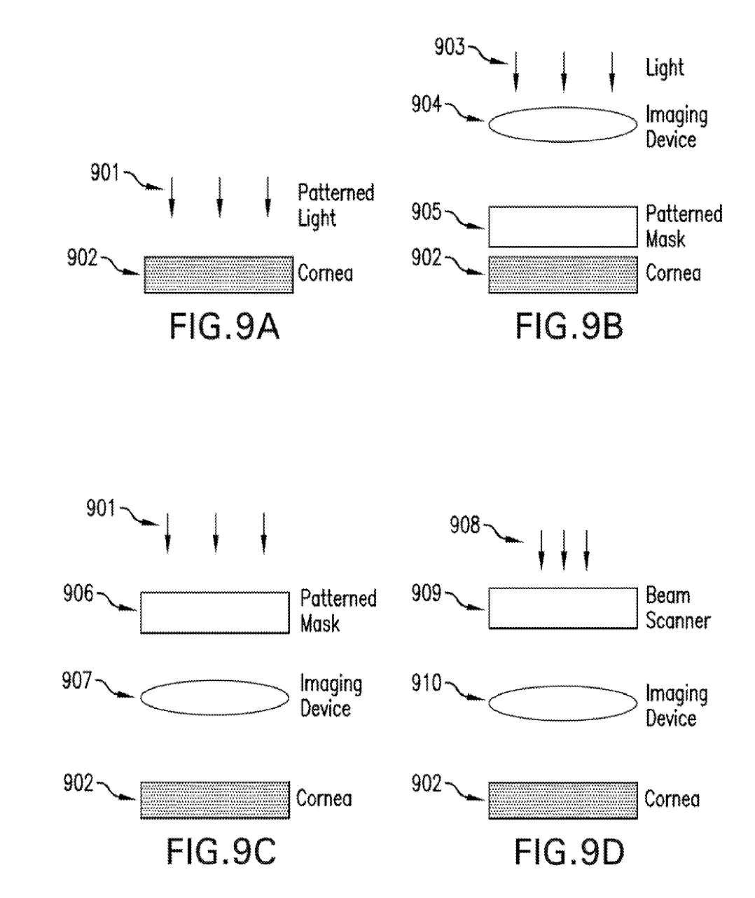

FIGS. 9A-9D illustrate a set of illustrations of various exemplary configurations of an application of electro-magnetic radiation with at least some portions of the exemplary apparatus for producing patterned epithelium debridement according to the exemplary embodiments of the present disclosure. In one exemplary embodiment shown in FIG. 9A, light or other electromagnetic radiation possessing a spatial pattern (901) is directly applied to the cornea sample (902). Several ways of patterning light (or other electromagnetic radiation) have been developed and could be applied. Alternatively, a shown in FIG. 9B, uniform light (903) (or other electromagnetic radiation) can be imaged onto the cornea sample through an imaging instrument (904), and a patterned mask (905) can be placed on top of a surface of the cornea sample (902). The same or a different patterned mask (906) can be also placed after the uniform light, such as in FIG. 9C, before an imaging device (907) to be projected onto the surface of the cornea sample (902). Alternatively or in addition, a collimated beam of light (908) (or other electromagnetic radiation) can be scanned onto the cornea sample (902) with a beam scanner (909) and/or an imaging device (910) to achieve a desired exemplary light pattern.

According to yet further exemplary embodiments of the present disclosure, the size of the small zones of removed epithelium can be varied. For example, the lower limit can be on the order of magnitude of the molecular size of the photosensitizing agent, and therefore the little holes can be as small as what diffraction-limited lasers can produce or even smaller, The upper limit for the size of the zones of epithelium removal is expected to be as big as, e.g., about 200 microns or more, which can be limited by the size that hinders the fast mode of re-epithelialization. Within these exemplary limits, the methods, arrangements and devices according to certain exemplary embodiments of the present disclosure can be customized and/or optimized.

For example, an estimation on exemplary improvements which can be associated with the methods, arrangements and devices according to exemplary embodiments of the present disclosure can be performed involving micro-injuries formation on skin through a technique, fractional photothermolysis, which is currently used with success for many purposes including scar removal and tissue rejuvenation. (See, e.g., Ref. 27). However, certain exemplary differences exist with respect to the skin application. For example, one such exemplary difference can relate to the depth of the treatment, e.g., in skin applications, the micro-holes or micro-injuries can usually run deep into the dermal layer, beyond the epithelium, because generally the intended purpose is tissue remodeling; on the other hand, according to an exemplary embodiment of the present disclosure, in the ocular tissue, only the epithelium needs to be removed, and removing deeper layers may represent a contra-indication.

As a result, the methods, arrangements and devices according to exemplary embodiments of the present disclosure are present which prevent a deeper injury. This can be done, e.g., by an exemplary optical engineering configuration and/or mechanically. For example, in skin, where micro-injuries are usually designed to be more significant, complete re-epithelialization can be observed in one day.

Thus, exemplary system, method and device according to the exemplary embodiments of the present disclosure can improve a post-op recovery time compared to current methods relying on macroscopic epithelial debridement. Further, in terms of yield of delivery of photosensitizer, if about 20-50% of surface area is open and accessible, performances comparable to macroscopic epithelial debridement can be achieved with minimal adjustments to the second step of the CXL procedure, i.e., the application/diffusion/soaking time of the photosensitizer, especially if the diffusion properties of the photosensitizer can be optimized for the intended application.

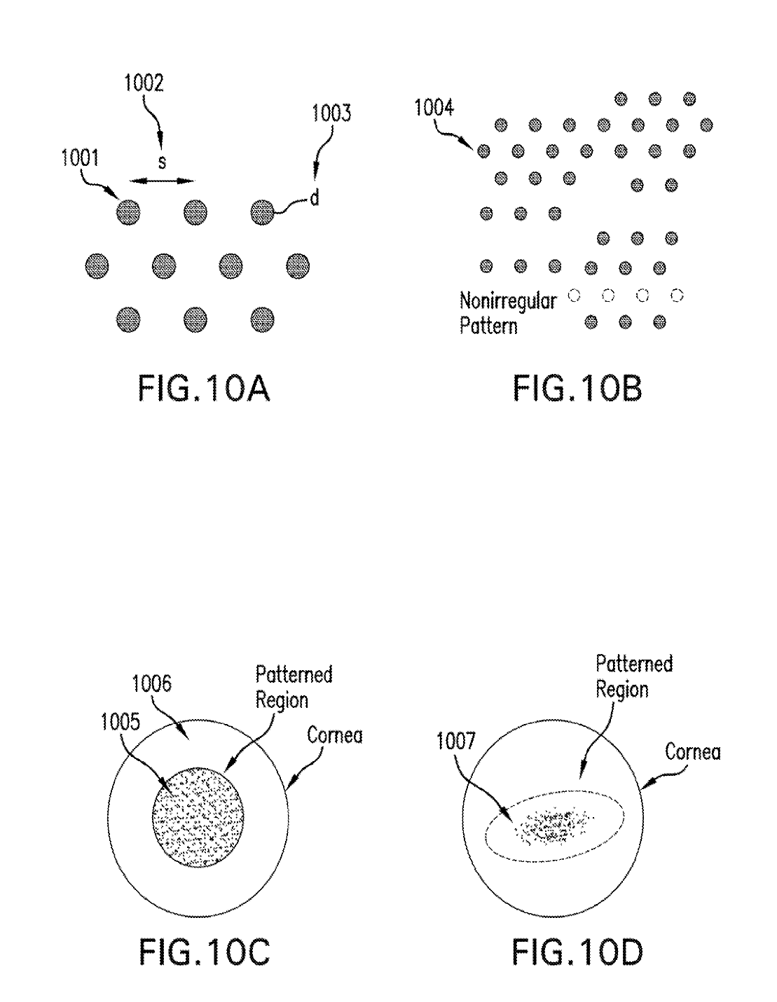

Further, the localized epithelium removal can be achieved, e.g., optically and/or mechanically. For example, different lasers or other light sources can be used for such purpose with varying pulsed duration, wavelength and/or energy (with much lower requirements with respect to the skin or other tissue application). Appropriate performances can be achievable because, e.g., a) in terms of retinal exposure safety, the natural divergence of the focused beam used to create the small holes can project a large unfocused beam onto the retina; and b) in terms of cornea thermal safety, only a small localized area of tissue is affected, the surrounding tissue can be unexposed and continuous perfusion of corneal tissue can be obtained through the aqueous humor. FIG. 10 illustrates sets of exemplary patterns used with the exemplary embodiments of the present disclosure for impacting the cornea, and the exemplary results of such applications in accordance with the exemplary embodiments of the present disclosure.

FIG. 10A shows for a regular, periodic debridment with holes (1001) created with a certain spacing s (1002) and diameter d (1003), according to exemplary embodiments of the present disclosure. These exemplary parameters can be important for an optimization of an exemplary procedure according to certain exemplary embodiments of the present disclosure in terms of, e.g., speed of healing, speed of the procedure, effectiveness of the drug diffusion, etc. In addition, irregular patterns (1004) can be created using such exemplary embodiments, as shown in, e.g., FIG. 10B, depending on the abnormality measured on the cornea and the desired outcome. FIG. 10C shows an exemplary spatial distribution of the treated area within the cornea. Patterning the holes and controlling their spacing and diameter, facilitates a regulation of the spatial diffusion of the photosensitizer, which can provide an accurate control of a treated area (1005) versus an untreated (1006) area. This exemplary also shown in FIG. 10D, which illustrates that the shape of the treated area (1007) and its effective dosing can be adjusted to form an ellipse with different areas of treatment. An analogous exemplary control of the diffusion can be obtained using mechanical devices such as, e.g., microneedles, miniaturized biopsy punches, etc.

Alternatives to optical arrangements/configurations, mechanical arrangements and/or configurations can be used. For example, in skin and other tissues, it has been recently shown that, similar healing/recovery performances to fractional photothermolysis can be obtained by a patterned array of sharpened micro-needles that extract ˜100-micron-sized columns of tissue. (See, e.g., Ref 28). This can represent a proof-of-principle for the exemplary embodiments of the present disclosure as showing that microscopic epithelium removal can be obtained mechanically in a controlled manner. This can be done, e.g., with patterned array of microneedles, robotically-driven scanned needles, and/or other devices that replicate the biopsies procedure at a microscopic scale and with more limited depth.

The foregoing merely illustrates the principles of the disclosure. Various modifications and alterations to the described embodiments will be apparent to those skilled in the art in view of the teachings herein. Indeed, the arrangements, systems and methods according to the exemplary embodiments of the present disclosure can be used with and/or implement any OCT system, OFDI system, SD-OCT system or other imaging systems, and for example with those described in International Patent Application PCT/US2004/029148, filed Sep. 8, 2004 which published as International Patent Publication No. WO 2005/047813 on May 26, 2005, U.S. patent application Ser. No. 11/266,779, filed Nov. 2, 2005 which published as U.S. Patent Publication No. 2006/0093276 on May 4, 2006, and U.S. patent application Ser. No. 10/501,276, filed Jul. 9, 2004 which published as U.S. Patent Publication No. 2005/0018201 on Jan. 27, 2005, and U.S. Patent Publication No. 2002/0122246, published on May 9, 2002, the disclosures of which are incorporated by reference herein in their entireties. It will thus be appreciated that those skilled in the art will be able to devise numerous systems, arrangements and methods which, although not explicitly shown or described herein, embody the principles of the disclosure and are thus within the spirit and scope of the present disclosure. It should be understood that the exemplary procedures described herein can be stored on any computer accessible medium, including a hard drive, RAM, ROM, removable disks, CD-ROM, memory sticks, etc., and executed by a processing arrangement and/or computing arrangement which can be and/or include a hardware processors, microprocessor, mini, macro, mainframe, etc., including a plurality and/or combination thereof. In addition, certain terms used in the present disclosure, including the specification, drawings and claims thereof, can be used synonymously in certain instances, including, but not limited to, e.g., data and information. It should be understood that, while these words, and/or other words that can be synonymous to one another, can be used synonymously herein, that there can be instances when such words can be intended to not be used synonymously. Further, to the extent that the prior art knowledge has not been explicitly incorporated by reference herein above, it can be explicitly incorporated herein in its entirety. All publications referenced herein can be incorporated herein by reference in their entireties.

### REFERENCES

1. Wollensak, G., Spoerl, E. & Seiler, T. Riboflavin/ultraviolet-a-induced collagen crosslinking for the treatment of keratoconus. American Journal of Ophthalmology 135, 620-627 (2003).
2. Hovakimyan, M., Guthoff, R. F. & Stachs, O. Collagen Cross-Linking-Current Status and Future Directions. Journal of Ophthalmology 2012, 1-12 (2012).
3. Schumacher, S., Mrochen, M., Wernli, J., Bueeler, M. & Seiler, T. Optimization Model for UV-Riboflavin Corneal Cross-linking Investigative Ophthalmology & Visual Science 53, 762-769 (2012).
4. McCall, A. S., et al. Mechanisms of Corneal Tissue Cross-linking in Response to Treatment with Topical Riboflavin and Long-Wavelength Ultraviolet Radiation (UVA). Investigative Ophthalmology & Visual Science 51, 129-138 (2010).
5. Sakimoto, T., Rosenblatt, M. I. & Azar, D. T. Laser eye surgery for refractive errors. The Lancet 367, 1432-1447.
6. Kochevar, I. E. & Redmond, R. W. Photosensitized production of singlet oxygen. in Methods in Enzymology, Vol. Volume 319 (ed. Lester Packer, H. S.) 20-28 (Academic Press, 2000).
7. Frederiksen, P. K., et al. Two-Photon Photosensitized Production of Singlet Oxygen in Water. Journal of the American Chemical Society 127, 255-269 (2004).
8. Baier, J., et al. Singlet Oxygen Generation by UVA Light Exposure of Endogenous Photosensitizers. Biophysical Journal 91, 1452-1459 (2006).
9. Helmchen, F. & Denk, W. Deep tissue two-photon microscopy. Nature Methods 2, 932-940 (2005).
10. Hafezi, F. Limitation of collagen cross-linking with hypoosmolar riboflavin solution: failure in an extremely thin cornea. Cornea 30, 917-919 (2011).
11. Scarcelli, G. & Yun, S. H. Confocal Brillouin microscopy for three-dimensional mechanical imaging. Nature Photonics 2, 39-43 (2008).
12. Scarcelli, G., Pineda, R. & Yun, S. H. Brillouin Optical Microscopy for Corneal Biomechanics. Investigative Ophthalmology & Visual Science 53, 185-190 (2012).
13. Olsen, T. On the calculation of power from curvature of the cornea. British Journal of Ophthalmology 70, 152-154 (1986).
14. Wollensak, G., Spoerl, E. & Seiler, T. Stress-strain measurements of human and porcine corneas after riboflavin-ultraviolet-A-induced cross-linking Journal of Cataract & Refractive Surgery 29, 1780-1785 (2003).
15. Scarcelli, G., et al. Brillouin Microscopy of Collagen Crosslinking: Noncontact Depth-Dependent Analysis of Corneal Elastic Modulus. Investigative Ophthalmology & Visual Science 54, 1418-1425 (2013).
16. Patterson, G. H. & Piston, D. W. Photobleaching in Two-Photon Excitation Microscopy. Biophysical Journal 78, 2159-2162 (2000).
17. Patel, S. V., Maguire, L. J., McLaren, J. W., Hodge, D. O. & Bourne, W. M. Femtosecond Laser versus Mechanical Microkeratome for LASIK: A Randomized Controlled Study. Ophthalmology 114, 1482-1490 (2007).
18. Yavuz, M. S., et al. Gold nanocages covered by smart polymers for controlled release with near-infrared light. Nature Materials 8, 935-939 (2009).
19. G. Wollensak, E. Spoerl, and T. Seiler, “Riboflavin/ultraviolet-a-induced collagen crosslinking for the treatment of keratoconus,” American Journal of Ophthalmology, vol. 135, pp. 620-627, 2003.
20. M. Hovakimyan, R. F. Guthoff, and O. Stachs, “Collagen Cross-Linking: Current Status and Future Directions,” Journal of Ophthalmology, vol. 2012, pp. 1-12, 2012.
21. K. M. Meek and S. Hayes, “Corneal cross-linking—a review,” Ophthalmic and Physiological Optics, vol. 33, pp. 78-93, March 2013.
22. G. Wollensak and E. Iomdina, “Biomechanical and histological changes after corneal crosslinking with and without epithelial debridement,” Journal of Cataract and Refractive Surgery, vol. 35, pp. 540-546, March 2009.
23. L. Yuen, C. Chan, and B. S. B. Wachler, “Effect of epithelial debridement in corneal collagen crosslinking therapy in porcine and human eyes,” Journal of Cataract and Refractive Surgery, vol. 34, pp. 1815-1816, November 2008.
24. N. M. Ziebarth, I. Persaud, E. Arrieta, J. Dias, E. Lee, C. Rowaan, I. Nose, S. H. Yoo, and J.-M. A. Parel, “Efficiency of Coulomb Controlled Iontophoresis for Transcorneal Delivery of Riboflavin: A pilot study,” ARVO Annual Meeting Abstract Search and Program Planner, vol. 2011, pp. 2544-2544, May 2013.
25. G. Scarcelli, S. Kling, E. Quijano, R. Pineda, S. Marcos, and S. H. Yun, “Brillouin Microscopy of Collagen Crosslinking: Noncontact Depth-Dependent Analysis of Corneal Elastic Modulus,” Investigative Ophthalmology & Visual Science, vol. 54, pp. 1418-1425, February 2013.
26. D. Manstein, G. S. Herron, R. K. Sink, H. Tanner, and R. R. Anderson, “Fractional photothermolysis: A new concept for cutaneous remodeling using microscopic patterns of thermal injury,” Lasers in Surgery and Medicine, vol. 34, pp. 426-438, 2004 2004.
27. E. P. Tierney, D. J. Kouba, and C. W. Hanke, “Review of Fractional Photothermolysis: Treatment Indications and Efficacy,” Dermatologic Surgery, vol. 35, pp. 1445-1461, October 2009.
28. E. Cheung, M. Yao, W. Franco, M. Purschke, J. Tam, W. Farinelli, Y. Wang, and R. R. Anderson, “LESSONS FROM FRACTIONAL PHOTOTHERMOLYSIS APPLIED TO TISSUE ENGINEERING AND WOUND HEALING: CAN SKIN BE “COPIED”?,” Lasers in Surgery and Medicine, vol. 44, pp. 353-353, April 2012.

What is claimed is:

1. An apparatus, comprising:
at least one computer first arrangement which is configured to:
a. obtain at least one property of at least one portion of the eye,
b. based on the at least one property, generate data indicating a plan of affecting the at least one portion of the eye, and
c. control at least one electromagnetic-radiation-providing second arrangement to execute the plan and irradiate the at least one portion based on the plan, wherein the irradiation is selectively controlled to be delivered to at least one selective depth within the at least one portion.

2. The apparatus according to claim 1, wherein the at least one further arrangement includes a laser source configured to excite multi-photon transitions.

3. The apparatus according to claim 2, wherein the laser source is a pulsed femto-second laser source which is configured to deliver a near infra-red light radiation.

4. The apparatus according to claim 1, wherein the at least one property obtained includes at least one of (i) refractive index, (ii) elastic or visco-elastic property, (iii) microstructure, (iv) radius of curvature, (v) collagen content and organization, or (vi) scattering effect of the at least one portion.

5. The apparatus according to claim 1, wherein the at least one property is obtained by at least one (i) OCT, (ii) Brillouin imaging modality (iii) Raman, (iv) laser speckle, (v) multi-photon imaging modality, (vi) photo-acoustic modality, (vii) confocal microscopy modality, (viii) florescence modality, (ix) pentacam, or (x) ultrasound imaging.

6. The apparatus according to claim 1, wherein the effects of the plan on at least one portion of the eye include at least one change in the at least one property.

7. The apparatus according to claim 6, wherein the at least one change includes a change to (i) a refractive index, (ii) an elastic or visco-elastic property, (iii) a microstructure, (iv) a radius of curvature, (v) a collagen content, or (vi) a scattering effect of the at least one portion.

8. The apparatus according to claim 7, wherein the at least one change effects at least one optical property of the eye.

9. The apparatus according to claim 8, wherein the at least one optical property includes a refractive error within the eye.

10. The apparatus according to claim 9, wherein the refractive error includes at least one of a myopia, a hyperopia, an astigmatism or a high-order aberration.

11. The apparatus according to claim 10, wherein the high-order aberration includes at least one a spherical aberration or a coma aberration.

12. The apparatus according to claim 8, wherein the at least one optical property includes at least one of (i) a refractive property, (ii) a transmission property, (iii) a polarization filter, (iv) a reflection property, or (v) a color filter.

13. The apparatus according to claim 1, wherein the irradiation is delivered to a specifically-controlled volume within the at least one portion.

14. The apparatus according to claim 7, wherein the specifically controlled volume comprises a spatially controlled pattern optimized to execute the plan based on the at least one property

15. The apparatus according to claim 1, wherein the irradiation is delivered to a specifically controlled volume within at least one portion without effecting further portions of the at least one portion through which the irradiation is delivered.

16. The apparatus according to claim 15, wherein the specifically controlled volume is as small as a diffraction-limited spot delivered by the second arrangement up to less than the volume of the at least one portion.

17. The apparatus according to claim 15, wherein the specifically controlled volume is approximately 1 micron³.

18. The apparatus according to claim 1, further comprising at least one third arrangement which is configured to effect a further property of the eye at least one of (i) prior to or (ii) during the delivery of the irradiation to the at least one portion.

19. The apparatus according to claim 18, wherein the at least one third arrangement is configured to applanate the cornea or counteract the intrinsic refractive power of the cornea to facilitate cross-linking, and wherein the at least one third arrangement includes at least one of (i) a contact lens (ii) a concave lens, (iii) a convex lens, (iv) an applanating transparent window or (v) a prism.

20. The apparatus according to claim 1, wherein the at least one portion contains a photo-activatable agent.

21. The apparatus according to claim 30, wherein the at least one first arrangement causes an activation of the photo-activatable agent so as to cause a selective cross-linking.

22. The apparatus according to claim 21, wherein the at least one first arrangement uses the selective cross-linking to treat keratoconus in the at least one portion.

23. The apparatus according to claim 21, wherein the at least one first arrangement obtains information regarding keratoconus in the at least one portion, and changes a refractive property of the at least one portion based the information using the selective cross-linking.

24. The apparatus according to claim 23, wherein the refractive property includes a high-order aberration.

25. The apparatus according to claim 24, wherein the high-order aberration includes at least one a spherical aberration or a coma aberration.

26. The apparatus according to claim 1, wherein, upon the execution of the plan and the delivery of the irradiation to the at least one portion based on the plan, at least one of (i) a refractive error or (ii) an imperfection of the eye is improved.

27. The apparatus according to claim 1, wherein, upon the execution of the plan and the delivery of the irradiation to the at least one portion based on the plan, at least one separation within the eye is reconnected.

28. The apparatus according to claim 1, wherein the reconnection includes a selective biomechanical treatment for flap bonding.

29. The apparatus according to claim 1, wherein the plan comprises ablating at least two electro-magnetic radiations to at least two first regions of epithelium of the eye which are separated by a unablated second region.

30. The apparatus according to claim 1, wherein the plan comprises penetrating at least at least two first regions of epithelium of the eye which are separated by an unpenetrated second region; and removing localized zones of an epithelial layer from the first regions.

31. The apparatus according to claim 1, wherein the irradiation is selectively controlled by the at least one first arrangement to provide a spatially-periodic pattern within the at least one portion.

32. A method comprising:
with at least one first arrangement, obtaining at least one property of at least one portion of the eye;
based on the at least one property, generating data indicating a plan of affecting the at least one portion of the eye; and
controlling at least one electromagnetic-radiation-providing second arrangement to execute the plan and irradiate the at least one portion based on the plan, wherein the irradiation is selectively controlled to be delivered to at least one selective depth within the at least one portion.

33. A non-transitory computer-accessible medium which includes executable instructions, wherein, when the executable instructions are executed by a computing arrangement, the computer arrangement is configured to execute procedures comprising:
with at least one first arrangement, obtaining at least one property of at least one portion of the eye;
based on the at least one property, generating data indicating a plan of affecting the at least one portion of the eye; and
controlling at least one electromagnetic-radiation-providing second arrangement to execute the plan and irradiate the at least one portion based on the plan, wherein the irradiation is selectively controlled to be delivered to at least one selective depth within the at least one portion.

34. An apparatus for treating an eye structure, comprising:
a delivery arrangement configured to direct an electromagnetic radiation generated by an electromagnetic radiation source to at least one particular area within a target area of epithelium of the eye structure, wherein the electromagnetic radiation is adapted to at least one of ablate or cause thermal damage to an epithelium layer of the eye from a surface of the skin through an entire depth of the epithelium layer; and
a control arrangement configured to interact with the delivery arrangement such that the delivery arrangement directs the electromagnetic radiation onto a plurality of spatially separated particular areas within the target area,
wherein the control arrangement is further configured such that, upon a completion of treatment of the entire target area, at least two immediately adjacent particular areas are separated from one another by at least one further epithelium section of the epithelium that is at least one of undamaged, unablated and/or unirradiated.

35. An apparatus for treating an eye structure, comprising:
a needle arrangement configured to direct at least one needle to at least one particular area within a target area of epithelium of the eye structure, so as to cause mechanical damage to an epithelium layer of the eye from a surface of the skin through an entire depth of the epithelium layer; and
a control arrangement configured to interact with the delivery arrangement such that the delivery arrangement controls the at least one needle to be inserted into a plurality of spatially separated particular areas within the target area,
wherein the control arrangement is further configured such that, upon a completion of treatment of the entire target area, at least two immediately adjacent particular areas are separated from one another by at least one further epithelium section of the epithelium that is undamaged.
